(19) United States
(12) Patent Application Publication
(10) Pub. No.: US 2006/0004332 A1
(43) Pub. Date: Jan. 5, 2006

(54) Vacuum installed external male catheters and methods of using same

(76) Inventor: Sherwood D. Marx, Tacoma, WA (US)

Correspondence Address: GRAYBEAL, JACKSON, HALEY LLP, 155 - 108TH AVENUE NE, SUITE 350, BELLEVUE, WA, 98004-5901, US

(21) Appl. No.: 10/884,642

(22) Filed: Jul. 2, 2004

(51) Int. Cl. A61F 5/44

(52) U.S. Cl. 604/349; 604/347

(57) ABSTRACT

Disclosed are male condom urinary catheters, methods for using the same and kits including instructions for mounting the catheters on a penis. The catheter has an interior surface and includes a thin cylindrical sheath of elastic material, an outlet configured for connection with a urine collection device, and a neck coupled between the sheath and the outlet. The instructions include coupling a vacuum source to the catheter, placing a circumferential portion the interior surface of the catheter proximate to a head of the penis, and applying a vacuum to the catheter sufficient to draw the circumferential portion of the catheter interior against the head of the penis. The instructions also include unrolling the catheter sheath rolls onto the penis, and releasing the vacuum. A treatment for male urinary dysfunction that includes wearing a sleeper catheter on a penis, and maintaining a substantially airtight closure of the sleeper catheter with the penis inside the catheter for an effective time.

### BACKGROUND

Urination incontinence devices with external catheters, also called urisheaths, are well known devices for gathering uncontrolled urine and flowing it into a collection device. Such devices also have well-known problems making them complicated, difficult, uncomfortable, and generally unreliable from the point of view of persons having to use them. One chronic problem is keeping the catheter in place on the penis, for which some such incontinence devices utilize an adhesive to removably bond the penis and the interior of the catheter. Some condom catheters are provided with an adhesive pre-applied to the interior of the catheter. Upon unrolling the condom catheter, the adhesive is positioned between the interior of the catheter and the penis and affixes the catheter to the penis. Catheters with pre-applied adhesive are difficult and expensive to manufacture. Other condom catheters are provided with separate adhesive strips. The patient first applies the adhesive strips to his penis, and then unrolls the catheter on his penis and over the adhesive strips, thus, affixing the catheter to the penis. Mounting a condom catheter tends to invoke a natural reaction of the penis to shrink and withdraw into the body. Furthermore, because catheters employ adhesives that are sticky by their nature, they tend to adhere where they first contact the penis, and are difficult to unroll and mount over a full length of a penis that is rapidly withdrawing into the body. This results in poorly mounted condom catheters that tend to spontaneously detach from the penis and/or leak. In addition, mounting a catheter to a penis with an adhesive causes discomfort to some patients over both a short term and a long term. Also, adhesives can irritate a penis.

Other existing devices first mount a condom catheter on a deployment tool or a housing assembly, and then use a vacuum to assist in positioning the catheter on a penis, or draw the penis into a housing. For example, applicant's prior U.S. Pat. No. 5,499,977 (incorporated herein by reference) utilizes a plastic housing as a deployment tool. The housing incorporates a rubber bulb as a vacuum source. The plastic housing includes a flared opening configured to match a penis tip. A rolled-up ring portion of a condom catheter is carried on the plastic housing, and deployed therefrom onto the penis. By way of further example, applicant's U.S. Pat. No. 5,662,631 (incorporated herein by reference) utilizes a vacuum to draw a penis having a previously mounted catheter into a tubular housing to which a urine drain tube is attached.

### SUMMARY

An embodiment of the present invention provides a method for quickly and comfortably mounting a condom catheter. The method comprises coupling a vacuum source to an outlet of a male condom urinary catheter, the catheter having an interior surface and including a thin cylindrical sheath of elastic material, the outlet configured for connection with a urine collection device, and a neck coupled between the sheath and the outlet. The method also comprises placing a portion of the interior surface of the catheter proximate to a head of a penis, at least a portion of the catheter sheath having been rolled outward upon itself to form consecutively larger rolls, applying a vacuum from the vacuum source to the catheter outlet sufficient to draw the portion of the interior of the catheter against the head of the penis, unrolling the catheter sheath onto the penis, and releasing the vacuum. The method may further comprise limiting movement of the head of the penis toward the wearer's body using the catheter after the catheter has been drawn against the penis. The method may also further comprise moving the head of the penis away from the wearer's body using the catheter after the catheter has been drawn against the penis.

A further aspect of the invention provides a sleeper catheter. The device comprises a generally cylindrical and at least semi-rigid catheter sleeve that includes an open proximal end having an inside diameter and an exterior surface, an open distal end, and a closure element that forms a substantially airtight seal in cooperation with the sleeve distal end, wherein the closure element is located at or proximate to the distal end. The sleeper catheter further comprises a thin cylindrical sheath of elastic material having an inside diameter less than the sleeve inside diameter. The sheath may be an integral extension of the sleeve or may be a separately joined element. If the sheath is removable from the sleeve, the sheath includes an open distal end configured for airtight coupling with a circumferential portion of the sleeve exterior surface. In either form, the sheath has an open proximal end configured for substantially airtight coupling with a penis, which forms a continuous interior chamber between the sheath proximal end and the sleeve distal end. The sleeve and the sheath cooperatively provide a substantially airtight installation on the penis by placing the open proximal end of the sheath closely around a circumference of the penis proximal to a head of the penis. The substantially airtight installation is further provided by applying a vacuum to the interior chamber sufficient to draw at least a portion of the penis and at least a portion of the sheath into the sleeve, and preferably bring the head of the penis proximate to the open distal end of the sleeve. The head of the penis is retained proximate to the distal end of the sleeve when the vacuum is released and the closure is applied to the open distal end of the sleeve such that, upon unintentional retraction of the penis from the sleeve, a vacuum is created biasing the penis toward the distal end. In an embodiment, the vacuum may be applied through the open distal end of the sleeve; the closure element may include a cap, and the open distal end of the sleeve correspondingly includes an engagement means for receiving the cap. The cap, when engaged with the open distal end, preferably does not extend more than 2.5 cm beyond the open distal end, thereby limiting the overall physical structure of the catheter.

An additional aspect of the invention provides a kit that comprises the sleeper catheter described above and instructions for mounting the sleeper catheter on a penis. The instructions comprise placing a circumferential portion of an interior surface of the sheath over a head of a penis and closely around a circumference of the penis proximal to a head of the penis. The instructions also comprise applying a vacuum to the interior chamber sufficient to draw at least a portion of the penis and at least a portion of the sheath into the sleeve and bring the head of the penis proximate to the open distal end of the sleeve, releasing the vacuum, and establishing a substantially airtight environment within the interior chamber. The instruction to establish a substantially airtight environment may include applying the closure element to the distal end of the sleeve, and may further include wearing the sleeper catheter.

Another aspect of the invention provides a method for treatment of male urinary dysfunction. The method comprises wearing a sleeper catheter on a penis, and maintaining the airtight closure of the sleeper catheter with the penis inside at least a portion of the sleeve of the device for an effective time duration. The male urinary dysfunction may include at least one condition from a group consisting of difficulty initiating the flow of urine, slow urine stream once urination has started, need to pass urine more often than usual during a day (frequency) or overnight (nocturia), dribbling, returning to urinate within a short time after initial urination, feeling of urgency to urinate, and sensation that emptying is not complete. The effective time duration may include wearing the device at least three hours a day for at least three days.

These and various other features as well as advantages of the present invention will be apparent from a reading of the following detailed description and a review of the associated drawings.

### BRIEF DESCRIPTION OF THE DRAWINGS

Aspects of the invention, together with features and advantages thereof, may best be understood by making reference to the following description taken in conjunction with the accompanying drawings, in the several figures of which like referenced numerals identify like elements, and wherein:

FIG. 1 illustrates a side and partial cross-section view of a male condom urinary catheter 20 of a kit, according to an embodiment of the invention;

FIG. 2 illustrates the catheter of FIG. 1 with the sheath in a rolled up configuration and a vacuum source, according to an embodiment of the invention;

FIG. 3 illustrates a head of a penis proximate to a circumferential portion of the interior surface of the catheter of FIG. 1, according to an embodiment of the invention;

FIG. 4 illustrates the sheath of the catheter of FIG. 1 unrolled onto the penis while the syringe maintains a vacuum, according to an embodiment of the invention;

FIG. 5 illustrates a partial cross-section view of a sleeper catheter, according to an embodiment of the invention;

FIG. 6 illustrates the sleeper catheter of FIG. 5 with a penis proximate to a circumferential portion of the interior surface of the sheath, according to an embodiment of the invention;

FIG. 7 illustrates the sleeper catheter of FIG. 5 with a vacuum source coupled to the distal opening, according to an embodiment of the invention;

FIG. 8 illustrates the sleeper catheter of FIG. 5 with the penis and a portion of the sheath having been drawn into a chamber formed by the interior surface of the sleeve, according to an embodiment of the invention; and

FIG. 9 illustrates the sleeper catheter of FIG. 5 with a closure element sealing the interior chamber and establishing an airtight environment, according to an embodiment of the invention.

### DETAILED DESCRIPTION

In the following detailed description of exemplary embodiments of the invention, reference is made to the accompanying drawings, which form a part hereof. The detailed description and the drawings illustrate specific exemplary embodiments by which the invention may be practiced. Other embodiments may be utilized, and other changes may be made, without departing from the spirit or scope of the present invention.

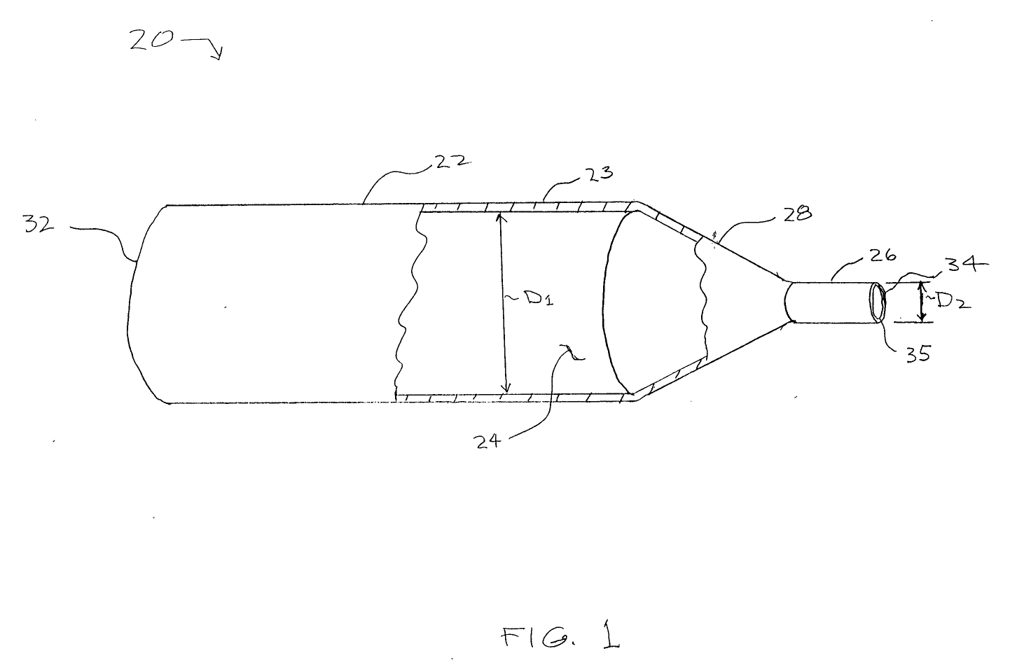

FIG. 1 illustrates a side and partial cross-section view of a male condom urinary catheter 20, according to an embodiment of the invention. The catheter 20 includes a cylindrical sheath 22 having a thin sidewall 23. The catheter 20 also includes an outlet 26 that connects with a gravity or vacuum urine collection device (not shown), and a neck 28 coupled between the sheath 22 and the outlet 26. The catheter 20 further includes a proximal end 32 of the sheath 22 that has an opening (shown in phantom) for insertion of a penis, and a distal end 34 having a urine discharge opening 35 that couples with the urine collection device. An interior surface 24 is defined between the proximal end 32 opening and the urine discharge opening 35.

The catheter 20 may have any configuration suitable for deployment and mounting on a penis, with or without use of an adhesive. For example, in one embodiment, the cylindrical sheath 22 includes a longitudinal length of approximately 7.5 cm and an internal diameter D₁ of approximately 2.5 cm. The outlet 26 includes a tubular structure with a length of approximately 2.5 cm, an outside diameter D₂ of approximately 1 cm, and a smaller inside diameter terminating in the discharge opening 35. The outlet 26 may have any configuration suitable for mounting with a urine collection device, such as the diameter D₂ being selected to sealingly fit with a tube leading into a urine collection bag. The neck 28 provides a dimensional, and optionally a structural, transition between the sheath 22 and the outlet 26. A portion of the interior surface 24 (not shown) of the neck 28 optionally provides a flexible and comfortable surface for contact with a head of a penis.

The catheter 20 may be made from any elastomer material suitable for direct contact with a penis, and may be a natural or synthetic material. For example, the catheter 20 may be made from a latex rubber, a silicone rubber, a polyester polyurethane, or a combination of these or other materials. The thin sidewall 23 may have any thickness suitable for condom catheter use that provides sufficient elasticity to elastically retain the sheath 22 on a penis over time; such thickness may be function of the material selected for the sheath 22. Optimally, the neck 28 and the outlet 26 have a thicker sidewall than the thin sidewall 23 of the sheath 22, providing at least a degree of rigidity or stiffness to these portions.

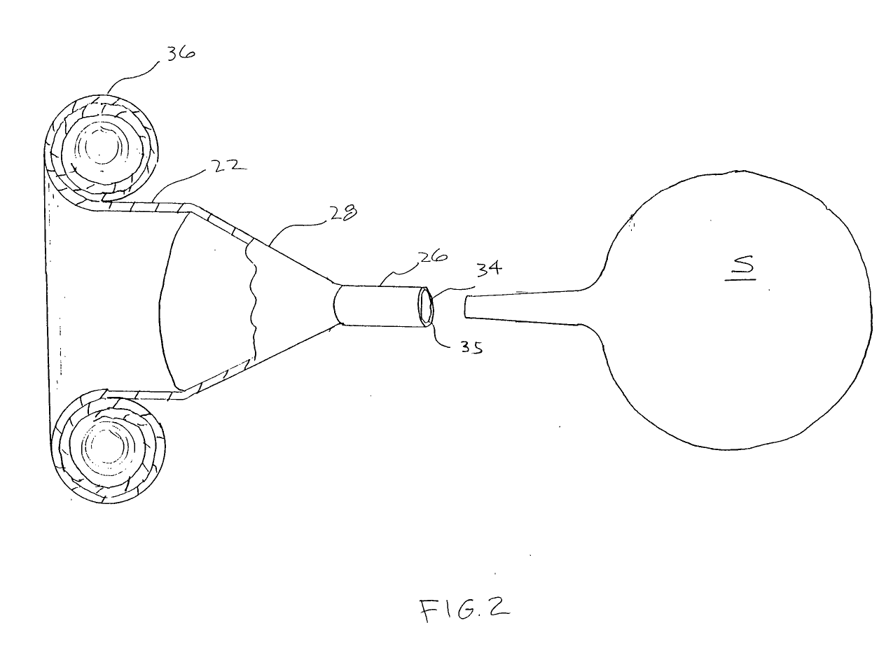

FIG. 2 illustrates the catheter 20 of FIG. 1 with the sheath 22 in a rolled up configuration and a vacuum source “S” in the form of a vinyl bulb, according to an embodiment of the invention. Before placement and/or mounting on a penis, at least a portion of the catheter sheath 22 is preferably rolled outward upon itself to form consecutively larger rolls 36. The catheter 20 is typically sold with at least a portion of the catheter sheath 22 rolled outward upon itself to form the consecutively larger rolls 36. If at least a portion of the catheter sheath 22 is not rolled into the rolls 36, a initial step in mounting the catheter 20 includes rolling the catheter sheath 22 outward upon itself starting at the proximal end 32 to form the consecutively larger rolls 36. The elasticity of the elastomer material retains the rolls 36 proximate to an unrolled portion of the sheath 22 or the neck 28. The rolls 36 form a funnel directing a penis into the unrolled portion of the sheath 22, and optionally proximate to the neck 28.

The vacuum source may provide any form of vacuum or suction, for example, the suction may be provided by a handheld device, a user inhaling through a tube, or a mechanical system. FIG. 2 illustrates the vacuum source as a handheld, soft vinyl bulb S, which is commonly available as an ear bulb. The vinyl bulb S includes a tapered inlet that sealingly fits inside the urine discharge opening 35.

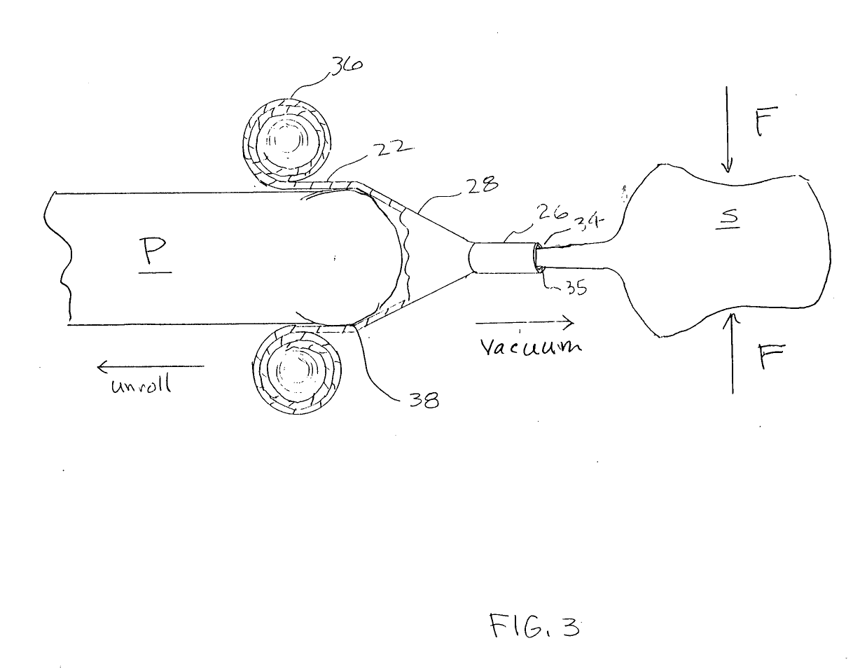

FIG. 3 illustrates a distal portion or head of a penis P proximate to a circumferential portion 38 of the interior surface 24 of the catheter 20 of FIGS. 1 and 2, according to an embodiment of the invention. FIG. 3 also illustrates the tapered inlet of the vinyl bulb S of FIG. 2 coupled into the discharge opening 35. A step in mounting the catheter 20 on a penis includes placing a circumferential portion of the penis P proximate to the circumferential portion 38 of the interior surface 24 of the catheter. In this step, a wearer will typically insert the head of the penis P past the catheter sheath roll 36 and proximate to the junction between the sheath 22 and the neck 28. The circumferential portion 38 may be at any location in either, or both of, the sheath 22 and the neck 28. The head of the penis P either may or may not, also, be proximate to the neck portion 28 of the catheter as a matter of personal preference. Optimally, a circumferential portion of the penis P contacts the circumferential portion 38 of the sheath 22 before application of the vacuum described below.

Another step in mounting the catheter 20 includes coupling the vacuum source, illustrated as the vinyl bulb S, to the distal opening 35 of catheter outlet 26. When the vacuum source is a handheld device such as the vinyl bulb S illustrated in FIG. 3, a compressive force F is applied to the vacuum source S to compress and ready it for application of a vacuum. The compressive force F may be applied at anytime prior to unrolling the rolls 36. For example, the vinyl bulb S may be first compressed and then coupled with the distal end 34. Alternatively, the vinyl bulb S may be loosely coupled with the distal end 34 and then compressed with the force F. Air forced from the vinyl bulb S is vented past the loose coupling. In another alternative, the vinyl bulb S is coupled with the distal end 34 and then compressed with the force F. Air forced from the vinyl bulb S is vented through a passage formed by the inner surface 24 and past the roll 36. If the head of the penis P has been placed proximate to the circumferential portion 38, the air is also vented past the head.

Another step in mounting the catheter 20 includes applying a vacuum from the vacuum source, illustrated as the vinyl bulb S, to the catheter outlet 26 sufficient to draw the circumferential portion 38 of the interior 24 of the catheter firmly against the head of the penis P and form a relatively airtight seal. In the embodiment illustrated in FIG. 3, the vacuum is applied by releasing the force F and allowing the body of the vinyl bulb S to expand toward its normal shape. The vacuum and airtight seal helps stabilize the catheter 20 relative to the head of the penis P and hold it in a relatively fixed position. Furthermore, the vacuum and resulting relatively airtight seal provide a grip on the head of the penis P by the catheter 20. A person may then hold the neck 28 of the catheter 20 firmly with their thumb and forefinger, and limit the natural shrink and withdraw movements of the head of the penis P toward the wearer's body by holding the catheter stationary relative to the body. Additionally, the grip also allows the penis P to be extended by gently moving the head of the penis P away from the wearer's body for improved mounting of the sheath 22. Both are expected to aid in mounting the condom catheter 20 on the penis P.

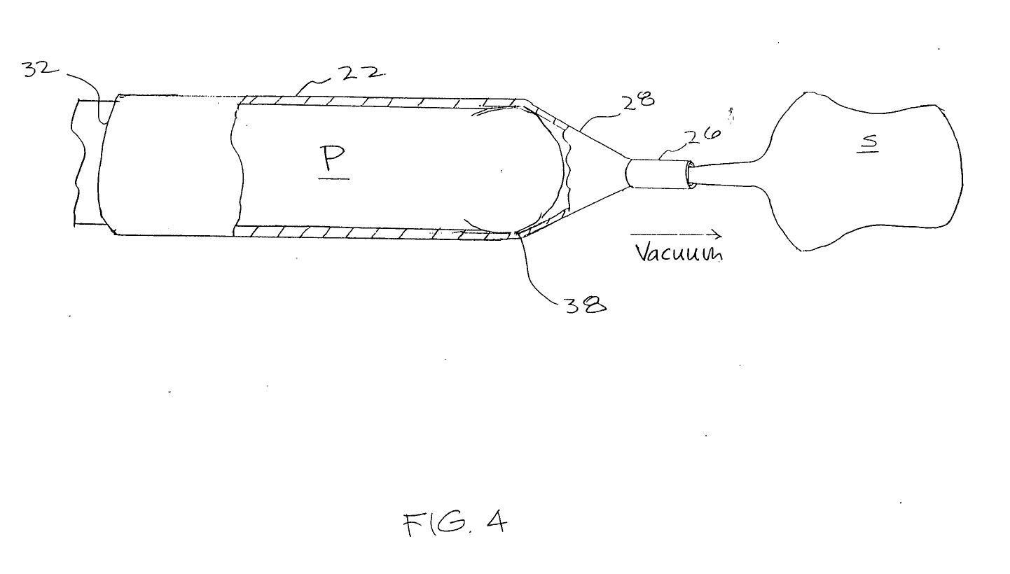

FIG. 4 illustrates the sheath 22 of the catheter 20 of FIGS. 1-3 unrolled onto the penis P while the vinyl bulb S maintains a vacuum, according to an embodiment of the invention. An additional mounting step includes unrolling the catheter sheath rolls 36 onto the penis P progressively away from the head of the penis. The grip provided by the vacuum may be used to stabilize the penis P during the unrolling of the catheter sheath rolls 36. As the sheath rolls 36 are unrolled, the thin sidewall 23 of sheath 22 is disposed proximate to the penis P. The elastic properties of the catheter sheath 22 reduce a circumference of the unrolled sheath against the penis P, drawing it elastically against the penis, retaining the sheath against the penis, and forming a further substantial airtight seal. The vacuum provided by the vinyl bulb S is maintained and/or renewed during the unrolling step, optimally until the entire roll 36 is unrolled.

A final step includes releasing the vacuum by uncoupling the vinyl bulb S from the catheter outlet. The sheath 22 remains against the penis P after the vacuum is released, and optimally the elasticity of the sheath maintains the airtight seal of the sheath 22 with penis P. The outlet 26 is ready for connection with a urine collection device. Such devices often apply a vacuum to the penis as an aid in collecting uncontrolled urine. Such vacuum is expected to reestablish a substantial airtight seal between the sheath 22 and the penis P if it was lost upon release of the vacuum. A substantially airtight seal over substantially an entire length of the penis P is expected to improve wearer comfort, protection against unexpected or premature need to change the catheter 20, and prevention of urine migration and build-up. The absence of an adhesive is further expected to improve these same factors.

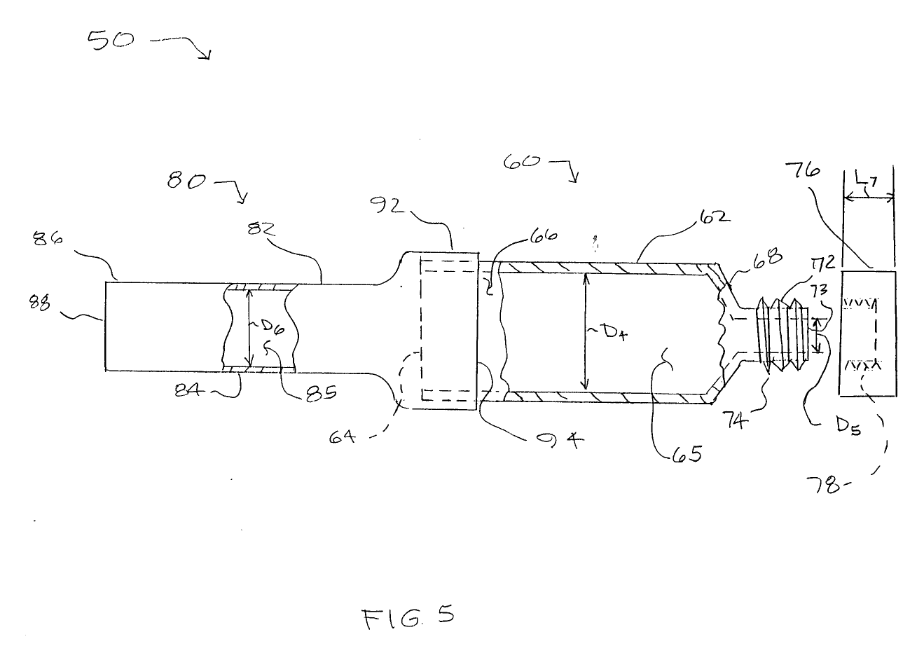

FIG. 5 illustrates a partial cross-section elevation view of a sleeper catheter 50, according to an embodiment of the invention. The sleeper catheter 50 includes a generally cylindrical and at least semi-rigid catheter sleeve 60, a closure element illustrated as a closure cap 76, and a condom sheath 80. The sleeve 60 includes a proximal portion 62 that has an opening 64, an inside diameter D₄, an interior surface 65, and an exterior surface 66. The sleeve 60 also includes a neck 68 coupling the proximal portion 62 and a distal end 72. The distal end 72 has an opening 73, inside diameter D₅, and a mount for the closure element, illustrated as threads 74 for a screw-on closure such as the closure cap 76. The closure cap 76 includes threads 78 that engage the threads 74 of the distal end 72. The interior surface 65 is defined between the proximal opening 64 and the distal opening 73.

The condom sheath 80 includes a thin cylindrical sheath 82 having an inside diameter D₆, an interior surface 85, a distal end portion 92 having an opening 94 configured for airtight coupling with a circumferential portion of the sleeve exterior surface 66. The inside diameter D₆ is less than the sleeve inside diameter D₄. The sheath 80 also includes a proximal end portion 86 having an opening 88 for insertion of a penis. The sheath 80 and the sleeve 60, when distal portion 92 is coupled with exterior surface 66, form a continuous interior chamber between the sheath proximal end opening 88 and the sleeve distal end opening 73.

The catheter sleeve 60 includes a tubular structure that is at least semi-rigid, and optimally thin walled for light weight. The sleeve 60 may be made from any suitable material, including a metal such as aluminum, a polymeric such as plastic, and a composite. The at least semi-rigid structure has sufficient rigidity and strength to maintain its shape while being worn as described below, and for the distal portion of the sleeve 80 to be compressively coupled to the exterior surface 66 of its proximal end portion 62. The neck 68 provides a dimensional and structural transition between the sleeve 60 and the distal end 72.

The condom sheath 80 is similar in materials and construction to the catheter 20 of FIG. 1, except that it has a generally uniform diameter D₆ and a single wall thickness. Further, like the catheter 20, the condom sheath may be made from any elastomer material suitable for direct contact with a penis, and may be either natural or synthetic material.

The closure element may be any device, or combination of devices, providing an airflow open position and sealed position. Since the sleeper catheter 50 may be worn during the day under normal clothing, an aspect of the invention provides an unobtrusive or low-profile closure element that minimizes visibility of the catheter under a man's clothes. Certain embodiments provide a low-profile closure cap 76 with threads 78 that engage threads 74 on the distal end 72, which seals the opening 73 when the threads are engaged. Other embodiments provide a toggled cap that toggles between an open position that does not obstruct the opening 73 and a closed position that seals the opening.

An embodiment of the sleeve 60 includes a hollow plastic cylinder having an internal diameter D₄ of approximately 3.5 cm, and a proximal end 62 length of approximately 10 cm. The sleeve 60 further includes a neck 68 and a distal end 72 having a length of approximately 1.3 cm each, and a distal end internal diameter D₅ of approximately 0.75 cm. An embodiment of the low-profile closure cap 76 includes a length L₇ of approximately 1.3 cm and course threads 74 that engage corresponding course threads 74 of the distal end 72.

An embodiment of the condom sheath 80 includes a thin-walled latex rubber sheath having a length of approximately 10 cm and an internal diameter D₆ of approximately 3.3 cm. The sleeve 60 and the sheath 80 are elastically coupled in an airtight configuration by stretching an approximately 1.3 cm length of the sheath 80 by hand over the proximal end opening 64 and further over approximately a 1.3 cm length of the exterior surface 66 of the proximal end portion 62 of the sleeve 60 as illustrated in FIG. 5. The coupling may be augmented in any manner, for example, by an adhesive, circular clamp, reinforced portion of the sheath 80, or a retention grove in the sleeve 50.

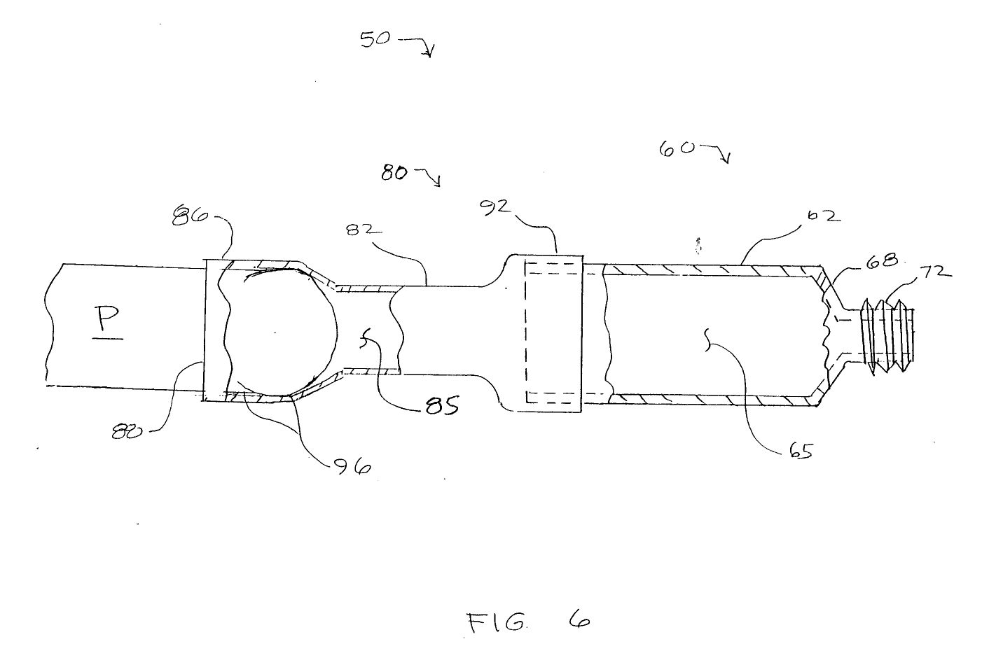

FIG. 6 illustrates the sleeper catheter 50 of FIG. 5 with a penis P proximate to a circumferential portion 96 of the interior surface 85 of the sheath 80, according to an embodiment of the invention. An initial step in mounting the sleeper catheter 50 on the penis P includes placing the open proximal end 88 of the sheath 82 around a circumferential portion of the penis proximate to the head of the penis. This may be accomplished by stretching the proximal opening 88 to enlarge its diameter, and moving it over the head of the penis and toward the body, and then releasing the stretched proximal opening, thus placing a circumferential portion 96 of the interior surface 85 of the sheath around a circumferential portion of the penis. The elasticity of the sheath 82 forms an airtight seal between the sheath and the penis.

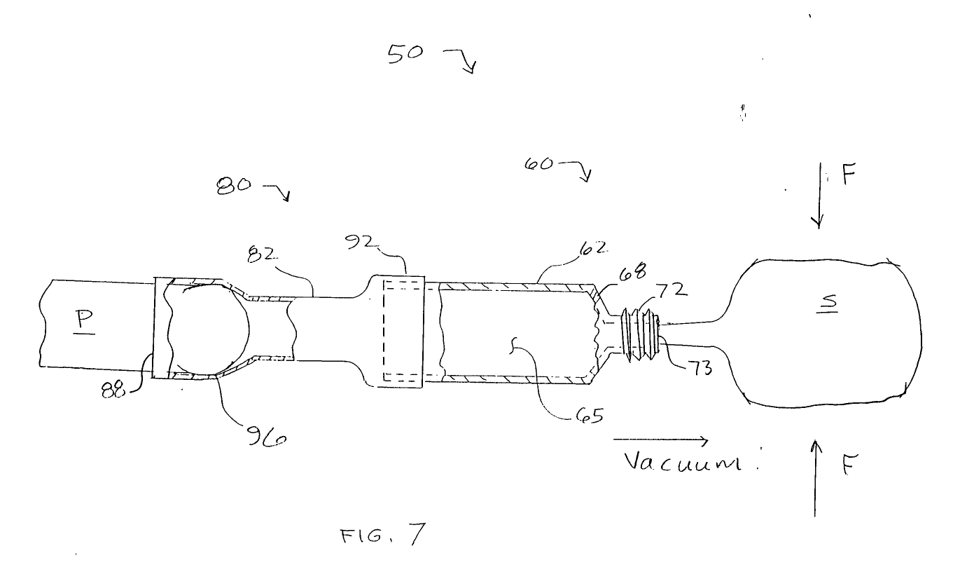

FIG. 7 illustrates the sleeper catheter 50 of FIG. 5 with a vacuum source coupled to the distal opening 73, according to an embodiment of the invention. Another step in mounting the sleeper catheter 50 on the penis P includes coupling a vacuum source to the catheter. As described in conjunction with the condom catheter 20 of FIGS. 1-4, the vacuum source may be any type of device or combination of devices or sources that provide a vacuum or suction. FIG. 7 illustrates the vacuum source as the vinyl bulb S of FIG. 2. Similarly, as described in conjunction with the condom catheter of FIGS. 1-4, a step in mounting the sleeper catheter 50 includes reading the vacuum source if required. When the vacuum source is the vinyl bulb S, it is readied for application of a vacuum by compressing it with the force F. Another step includes coupling the vacuum source, illustrated as vacuum source vinyl bulb S, to the sleeper catheter 50. FIG. 7 illustrates an embodiment where the vacuum source vinyl bulb S is coupled with the catheter 50 by inserting an outlet of the vinyl bulb into the distal end opening 73. In another embodiment, a separate sealable port for application of a vacuum is provided.

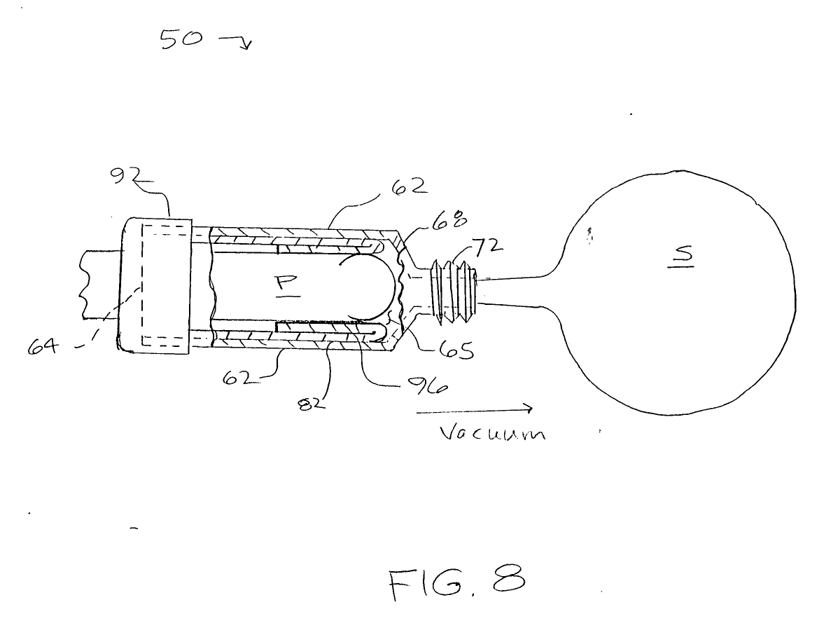

FIG. 8 illustrates the sleeper catheter 50 of FIG. 5 with the penis P and a portion of the sheath 80 having been drawn into a chamber formed by the interior surface 65 of the sleeve 60, according to an embodiment of the invention. A step in mounting the sleeper catheter 50 includes applying a vacuum to the interior chamber sufficient to draw at least a portion of the penis P and at least a portion of the sheath 80 into the sleeve 60 and bring the head of the penis proximate to neck 68 and/or the open distal end 73 of the sleeve. Typically, the head of the penis P is brought up into the proximal end 62 and within 0.6 to 1.3 cm of the neck 68.

Another step includes releasing the vacuum. The vacuum is released from the embodiment illustrated in FIG. 8 by uncoupling the vinyl bulb S from the distal end 73. The circumferential portion 96 of the condom sheath 80 remains coupled or around the circumferential portion of the penis P, and another portion of the sheath has been folded over the circumferential portion 96 with its elasticity reinforcing the airtight seal created with the penis by the circumferential portion 96. The head of the penis P is generally retained proximate to neck 68 and/or the open distal end 73 of the sleeve after the vacuum is released.

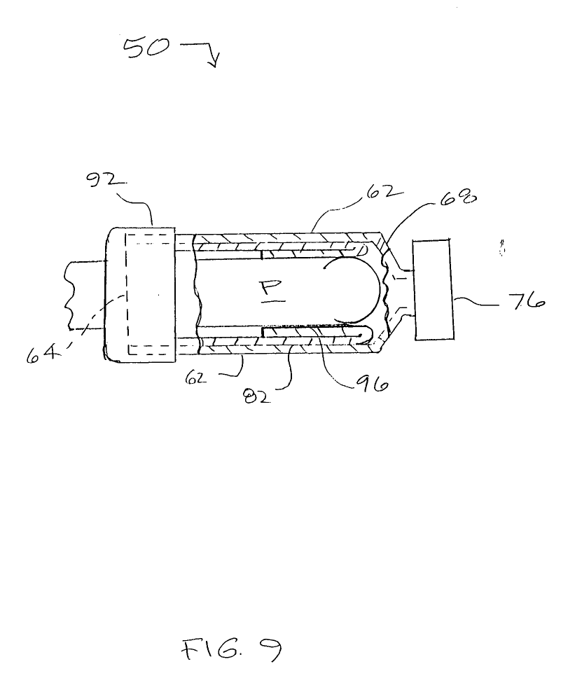

FIG. 9 illustrates the sleeper catheter 50 of FIG. 5 with a closure element sealing the interior chamber and establishing an airtight environment, according to an embodiment of the invention. The embodiment illustrated includes the closure cap 76 with threads that engage the distal threads 74 of the sleeve 60. A final step includes applying the closure element after release of the vacuum, which as illustrated, includes screwing the cap 76 onto the threads 74 until an airtight seal is formed. Application of the closure element establishes a sealed environment in cooperation with the substantially airtight seal by the circumferential portion 96 of sheath 82 against the penis P. The substantially sealed environment resists retraction, withdrawal, and/or shrinkage of the penis P from the catheter sleeve 50. Upon unintentional retraction of the penis P from the sleeve 50, a vacuum is created biasing the penis toward the neck 68 and distal end 72. Because the cap 76 has a low profile, such as a length L₇ of 2.5 cm or less, the sleeper catheter 50 has an overall small longitudinal profile and a reduced likelihood of being noticed while worn under clothes.

An aspect of the invention allows a wearer to urinate without removing the sleeper catheter 50. With an appropriately sized distal end inside diameter D₅, for example, a diameter of at least 0.6 cm, a wearer may unscrew the cap 76, urinate, clean the catheter distal end 72, and screw the cap 76 back on.

A kit according to an embodiment of the invention includes the sleeper catheter 50 and instructions for mounting the catheter on a penis. The instructions include steps that generally may be performed in any order except where specifically required. The instruction include:
placing a circumferential portion 96 of an interior surface 85 of the sheath 82 over a head of a penis and closely around a circumference of the penis proximal to the head of the penis;
applying a vacuum to the interior chamber formed by the interior surface 85 of the sheath 82 and the interior surface 65 of the sleeve sufficient to draw at least a portion of the penis and at least a portion of the sheath 82 into the sleeve and bring the head of the penis proximate to the open distal end 72 of the sleeve;
releasing the vacuum; and
establishing a substantially airtight environment within the interior chamber.

In another embodiment, a kit according to an embodiment includes the catheter sleeve 60 and instructions for mounting the condom sheath 80 on the sleeve to form the sleeper catheter and mounting the sleeper catheter on a penis. The instructions include:
coupling a circumferential portion of the sleeve exterior surface 66 to the open distal end 94 of the cylindrical sheath 80, the sheath and the sleeve when coupled forming a continuous interior chamber between the sheath proximal end and the sleeve distal end;
placing a circumferential portion 96 of an interior surface 85 of the sheath 82 over a head of a penis and closely around a circumference of the penis proximal to the head of the penis;
applying a vacuum to the interior chamber formed by the interior surface 85 of the sheath 82 and the interior surface 65 of the sleeve sufficient to draw at least a portion of the penis and at least a portion of the sheath 82 into the sleeve and bring the head of the penis proximate to the open distal end 72 of the sleeve;
releasing the vacuum; and
establishing a substantially airtight environment within the interior chamber.

In a further embodiment, a kit according to an embodiment of the invention includes a condom sheath 80 and instructions for mounting the condom sheath on the catheter sleeve 60 to form the sleeper catheter and mounting the sleeper catheter on a penis. The instructions include:
coupling a circumferential portion of the sleeve exterior surface 66 to the open distal end 94 of the cylindrical sheath 80, the sheath and the sleeve when coupled forming a continuous interior chamber between the sheath proximal end and the sleeve distal end;
placing a circumferential portion 96 of an interior surface 85 of the sheath 82 over a head of a penis and closely around a circumference of the penis proximal to the head of the penis;
applying a vacuum to the interior chamber formed by the interior surface 85 of the sheath 82 and the interior surface 65 of the sleeve sufficient to draw at least a portion of the penis and at least a portion of the sheath 82 into the sleeve and bring the head of the penis proximate to the open distal end 72 of the sleeve;
releasing the vacuum; and
establishing a substantially airtight environment within the interior chamber.
   Therapeutic Method

Urinary dysfunction includes conditions such as difficulty initiating a flow of urine, slow urine stream once urination has started, need to pass urine more often than usual during a day (frequency) or overnight (nocturia), dribbling, returning to urinate within a short time after initial urination, feeling of urgency to urinate, and sensation that emptying is not complete. Current options for treating urinary dysfunction are generally limited to medication and surgery, which may have adverse consequences.

Anecdotal trials done by the inventor on himself and others suggest that wearing the sleeper catheter 50 during at least a portion of a day for at least several days has a desirable effect of diminishing symptoms of male urinary dysfunction without adverse consequences to their health. For example, after wearing the sleeper catheter 50 during the day for several days, several individuals reported that their symptoms of urinary dysfunction diminished an observable amount, such as waking less often during a night to urinate, and reduced urination frequency during a day. The therapeutic duration, i.e., the number of hours the sleeper catheter 50 is worn per day and total number of days worn may vary depending on the individual and his particular symptoms. A minimum effective or therapeutic time duration is presently estimated at approximately at least three hours a day for at least three days. The sealed environment may be opened occasionally while the sleeper catheter 50 is being worn for urination or other reasons without adverse impact on the treatment. Since wearing the sleeper catheter 50 is not known to have any adverse health consequences, an individual may wish to wear the sleeper catheter as often as comfortable and/or convenient until a desired improvement in urinary function is achieved.

An embodiment of the invention provides a method for treatment of male urinary dysfunction. The method includes mounting the sleeper catheter 50 on a penis according to the instructions described above for an effective time duration for reduction of urinary dysfunction symptoms, such as approximately least three hours a day for at least three days.

Although the present invention has been described in considerable detail with reference to certain preferred embodiments, other embodiments are possible. Therefore, the spirit and scope of the appended claims should not be limited to the embodiment described herein.

What is claimed is:

1. A method for applying a male condom urinary catheter comprising:
coupling a vacuum source to an outlet of a male condom urinary catheter, the catheter comprising an interior surface and including a thin cylindrical sheath of elastic material, the outlet configured for connection with a urine collection device, and a neck coupled between the sheath and the outlet;
placing a portion of the interior surface of the catheter proximate to a head of a penis, at least a portion of the catheter sheath having been rolled outward upon itself to form consecutively larger rolls;
applying a vacuum from the vacuum source to the catheter outlet sufficient to draw the portion of the interior of the catheter against the head of the penis;
unrolling the catheter sheath onto the penis; and
releasing the vacuum.

2. The method of claim 1 further including limiting movement of the head of the penis toward the wearer's body using the catheter after the catheter has been drawn against the penis.

3. The method of claim 2 further include grasping the catheter and moving the head of the penis away from the wearer's body using the catheter after the catheter has been drawn against the penis.

4. The method of claim 1, wherein the catheter does not include an adhesive.

5. The method of claim 1, wherein the method does not include applying an adhesive to the penis.

6. The method of claim 1, further comprising the step of coupling the catheter outlet to an inlet of a urine collection device.

7. A male external catheter device comprising:
a generally cylindrical and at least semi-rigid catheter sleeve that includes an open proximal end having an inside diameter and an exterior surface, and an open distal end;
a closure element that forms an airtight seal in cooperation with the sleeve distal end, wherein the closure element is located at one of the distal end or proximate to the distal end; and
a thin cylindrical sheath of elastic material having an inside diameter less than the sleeve inside diameter, the sheath including an open distal end configured for airtight coupling with a circumferential portion of the sleeve, and an open proximal end configured for substantially airtight coupling with a penis, the sheath and the sleeve when coupled forming a continuous interior chamber between the sheath proximal end and the sleeve distal end,
the sleeve and the sheath cooperatively providing a substantially airtight installation on the penis by placing the open proximal end of the sheath closely around a circumference of the penis proximal to a head of the penis, applying a vacuum to the interior chamber sufficient to draw at least a portion of the penis and at least a portion of the sheath into the sleeve and bring the head of the penis proximate to the open distal end of the sleeve, and retain the head of the penis proximate to the distal end of the sleeve when the vacuum is released and the closure is applied to the open distal end of the sleeve such that, upon unintentional retraction of the penis from the sleeve, a vacuum is created biasing the penis toward the distal end.

8. The device of claim 7, wherein the vacuum is applied through the open distal end of the sleeve.

9. The device of claim 7, wherein the closure element includes a cap and the open distal end of the sleeve includes means for engaging the cap.

10. The device of claim 9, wherein the cap, when engaged with the open distal end, does not extend more than 2.5 cm beyond the open distal end.

11. A male external catheter device comprising:
a generally cylindrical and at least semi-rigid catheter sleeve that includes an open proximal end having an inside diameter and an exterior surface, and an open distal end, a circumferential portion of the sleeve exterior surface allowing a substantially airtight coupling with an open distal end of a sheath, the sheath including a cylinder of a thin elastic material and having an inside diameter less than the sleeve inside diameter, an open distal end allowing airtight coupling with the circumferential portion of the sleeve exterior surface, and an open proximal end allowing an airtight coupling with a penis, the sheath and the sleeve when coupled forming a continuous interior chamber between the sheath proximal end and the sleeve distal end; and
a closure element that forms a substantially airtight seal in cooperation with the sleeve distal end, wherein the closure element is located at one of the distal end or proximate to the distal end,
the sleeve and the sheath when coupled cooperatively providing a substantially airtight installation on the penis by placing the open proximal end of the sheath closely around a circumference of the penis proximal to a head of the penis, applying a vacuum to the interior chamber sufficient to draw at least a portion of the penis and at least a portion of the sheath into the sleeve and bring the head of the penis proximate to the open distal end of the sleeve, and retain the head of the penis proximate to the distal end of the sleeve when the vacuum is released and the closure is applied to the distal end of the sleeve such that, upon unintentional retraction of the penis from the sleeve, a vacuum is created biasing the penis toward the distal end.

12. A kit comprising:
a sleeper catheter according to claim 7; and
instructions comprising:
placing a circumferential portion of an interior surface of the sheath over a head of a penis and closely around a circumference of the penis proximal to a head of the penis;
applying a vacuum to the interior chamber sufficient to draw at least a portion of the penis and at least a portion of the sheath into the sleeve and bring the head of the penis proximate to the open distal end of the sleeve;
releasing the vacuum; and
establishing a substantially airtight environment within the interior chamber.

13. The kit of claim 12, wherein the instruction to establish a a substantially airtight environment includes applying the closure element to the distal end of the sleeve.

14. The kit of claim 12, wherein the instructions further include wearing the sleeper catheter.

15. A kit comprising:
a catheter sleeve and closure element according to claim 11; and
instructions comprising:
coupling a circumferential portion of the sleeve exterior surface to the open distal end of the thin cylindrical sheath of elastic material;
placing a circumferential portion of an interior surface of the sheath over a head of a penis and closely around a circumference of the penis proximal to the head of the penis;
applying a vacuum to the interior chamber sufficient to draw at least a portion of the penis and at least a portion of the sheath into the sleeve and bring the head of the penis proximate to the open distal end of the sleeve;
releasing the vacuum; and
establishing a substantially airtight environment within the interior chamber.

16. A method for treatment of male urinary dysfunction comprising wearing the device of claim 7 on a penis and maintaining the airtight closure with the penis inside at least a portion of the sleeve of the device for an effective time duration.

17. The method of claim 16, wherein the male urinary dysfunction includes at least one condition from a group consisting of difficulty initiating the flow of urine, slow urine stream once urination has started, need to pass urine more often than usual during a day (frequency) or overnight (nocturia), dribbling, returning to urinate within a short time after initial urination, feeling of urgency to urinate, and sensation that emptying is not complete.

18. The method of claim 16, wherein the effective time duration includes wearing the device at least three hours a day for at least three days.
